(19) United States
(12) Patent Application Publication
(10) Pub. No.: US 2003/0148517 A1
(43) Pub. Date: Aug. 7, 2003

(54) Preparation and compositions for antrodia camphorata mycelium biologically active material

(76) Inventors: Jinn-Chu Chen, Hsinchu City (TW); Chin-Nung Chen, Taoyuan City (TW); Sen-Je Sheu, Taiping City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 10/026,791

(22) Filed: Dec. 27, 2001

(51) Int. Cl. C12N 5/00; C12N 5/02; C12N 1/14; A61K 35/84; C12N 1/16; C12N 1/18

(52) U.S. Cl. 435/383; 435/254.1; 424/195.15

(57) ABSTRACT

The present invention relates to biologically active material, containing mainly polysaccharides, from the solution culturing for mycelium of Antrodia camphorata, a kind of mushroom that only grows inside a unique Taiwanese plant called Cinnamomum kanehirae tree, being able to improve immunity and resist tumors and parasites, and the preparation and compositions for the said active material.

### DESCRIPTION OF THE INVENTION

Types of Antrodia camphorate

Antrodia camphorata is also called Cinnamomum kanehirae mushroom, camphor mushroom, camphor chamber mushroom and yin-yang mushroom in Taiwan. The fruit body of Antrodia camphorata is perennial and has a strong smell. It differs a lot from general reishi mushroom in its plate-shaped or bell-shaped appearance. The plate-shaped one is orange red (yellow) with ostioles all over its surface and has light yellow white phellem in bottom layer. It grows by adhering phellem to the inner wall inside a hollow Antrodia camphorata. The bell-shaped one also shows orange (yellow) color in fruit body layer (bell surface) that is completely filled with ostioles (4˜5 ostioles/mm), inside, which are, spores of bitter taste in orange red for fresh state and in orange brown or brown afterward. Bell body is a shell that appears in dark green brown color. The spores look smooth and transparent in slightly curved column shape under the investigation by microscope.

Biological Characteristics of Antrodia amphorata

Wild Antrodia camphorata grows on the inner wall inside hollow Cinnamomum kanehirae tree. Because of this, many Cinnamomum kanehirae trees lie on the ground. According to literatures, Antrodia camphorata is the only rotten cunninghamia fungus ever found. It appears brown and rotten, so it is called rotten brown fungus. But Antrodia camphorata does not cause serious disease, so Cinnamomum kanehirae trees seldom die because of it. Although Antrodia camphorata is a kind of pathogenic bacterium to Cinnamomum kanehirae trees, its expensive price overpasses its economic value. Does it mean this pathogenic bacterium of Cinnamomum kanehirae trees is not important anymore?

The Culture of Antrodia camphorata

The culture of Antrodia camphorata still needs to be improved. So far, it is still collected from mountain field. However, the collection is a tough job. The first thing is to find where the Cinnamomum kanehirae trees are. The problem lies in the difficulty in distinguishing Cinnamomum kanehirae tree from micranthum hayata. The most direct method presently was proposed by [symbol] . Micranthum hayata tree oil is mainly composed of safrole and pentadecaldehyde, so it contains safrole smell in root beer. Cinnamomum kanehirae tree oil is mainly d-terpinenol, which smells like camphor oil. Hence the different smells are used to distinguish them. The second problem is to find the hollow trees in a large forest. This is very difficult. If Antrodia camphorata is found in the hollow Cinnamomum kanehirae tree, regular collection becomes possible.

Because it is hard to find hollow Cinnamomum kanehirae trees, unworthy businessmen cut down the trees for Antrodia camphorata to grow and collect it for sale. Therefore, under the consideration of environmental protection and economics, it is necessary to develop culturing technology for Antrodia camphorata. But there is never a technical breakthrough. Antrodia camphorata on Cinnamomum kanehirae wood chips grows slowly and even stops growth. Hence, using modem biotechnology to grow Antrodia camphorata mycelium will be the most economical and environmental protection compliant artificial culture.

Medical Effect and Active Ingredients for Antrodia camphorata

In an early legend, it is said the aboriginals in Taiwan happened to see Antrodia camphorata on Cinnamomum kanehirae trees when they were cutting and collecting plants in woods. The life style of the aboriginals tends to consume much body energy, so liver disease becomes their big threat. Besides due to the nature of the aboriginals, they like drinking very much, which increases the possibility of liver disease. However when they drink cooked Antrodia camphorata solution, they are healed soon and get strong. They believe Antrodia camphorata solution is very good to decompose alcohol. So the aboriginals consider Antrodia camphorata as a treasure and a traditional precious medicine. Some legends also said Antrodia camphorata could heal liver cancer, uterus cancer and even acute abdominal pain. There is not much scientific study on this subject. The School of Pharmacy in National Taiwan University has found apparent toxication to mouse malignant lymphocytic cells P-388. Taiwan Normal University pointed out that it has the functions like anti-choline, stool relaxation and blood platelet aggregation. Besides, it can inhibit the growth of staphylococcus aureaus and trichophyton mentagrophytes.

In Views of the Following:

1. The only specie that Antrodia camphorata can grow with parasitism is Cinnamomum kanehirae tree, which is under protection by laws. Besides, hollow Cinnamomum kanehirae trees are difficult to find.

2. There exist difficulties to grow Antrodia camphorata in vitro and exterior to Cinnamomum kanehirae trees.

3. Antrodia camphorata mycelium has virtual biological function and it is possible to carry out the culture and scale up the production.

The inventor of the present invention has spent tremendous efforts in research and found that both the culture solution and mycelium from Antrodia camphorata contained biologically active material. Accordingly, the present invention is accomplished.

### BRIEF DESCRIPTION OF THE DRAWINGS

FIG. 1 shows, according to CCRC 35398 culture and process of producing biologically active material from Antrodia camphorata mycelium in the present invention, the variations of the yields of Antrodia camphorata mycelium and the obtained biologically active material, i.e. polysaccharides, in dry weight percentage with the culturing time;

FIG. 2 shows, according to CCRC 35396 culture and process of producing biologically active material from Antrodia camphorata mycelium in the present invention, the variations of the yields of Antrodia camphorata mycelium and the obtained biologically active material, i.e. polysaccharides, in dry weight percentage with the culturing time;

FIG. 3 is the protein standard curve for gel filtration chromatography;

FIG. 4 is the chromatographic curve for molecular weight determination for polysaccharides contained in Antrodia camphorata mycelium;

FIG. 5 is the chromatographic curve for molecular weight determination for Sepharose 6B polysaccharides extracted from Antrodia camphorata mycelium by water;

FIG. 6 is the chromatographic curve for molecular weight determination for Sepharose 6B polysaccharides extracted from Antrodia camphorata mycelium by bases;

FIG. 7 is the ¹H-NMR spectrum for Sepharose 6B polysaccharides extracted from Antrodia camphorata mycelium by water;

FIG. 8 is the 13C-NMR spectrum for Sepharose 6B polysaccharides extracted from Antrodia camphorata mycelium by water;

FIG. 9 is the IR spectrum for polysaccharides from Antrodia camphorata mycelium; FIG. 10 is the X-ray diffraction patterns for polysaccharides from Antrodia camphorata mycelium;

FIG. 11 shows the variation of TNF-alpha concentrations from macrophage and analyzed by ELISA with different polysaccharide preparations when water-extracted material and base-extracted material from the Antrodia camphorata mycelium in the present invention and the fermentation solution undergo macrophage activation tests.

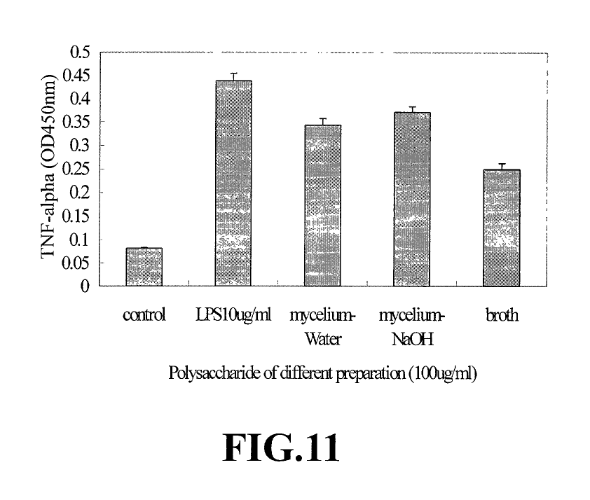

FIG. 12 shows the results of immune responses (cytokines IL-2, TNT-α and INF-γ) from C57BL/6 and BALB/c mice fed with different dosages of Antrodia camphorata for different number of weeks.

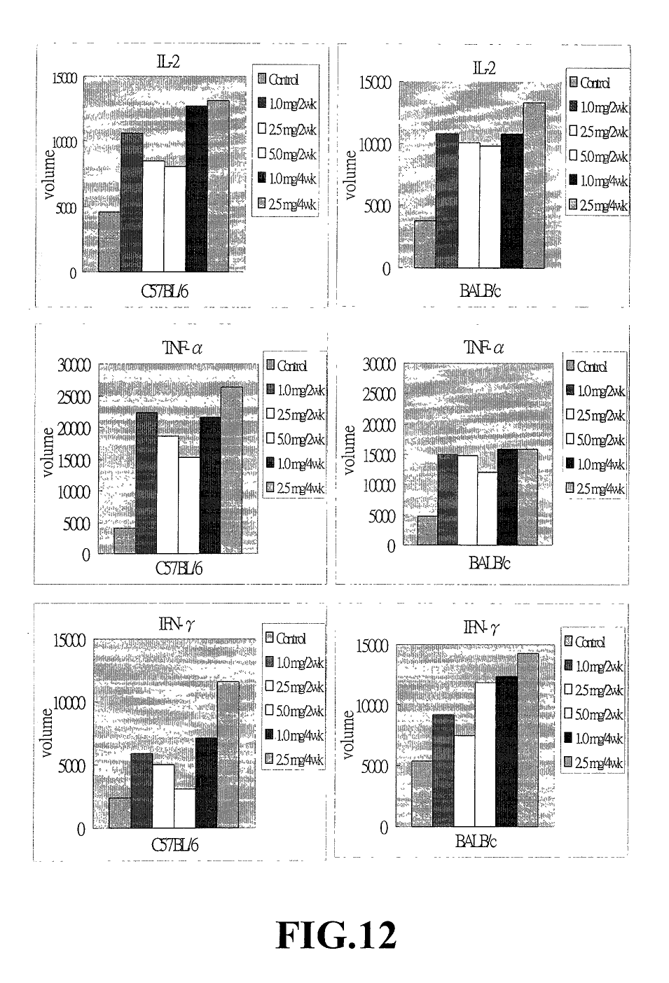

To summarize the above description, the present invention provides a kind of biologically active material from Antrodia camphorata, which is cultured in Antrodia camphorata mycelium solution and separated from the culture solution and/or mycelium, and is a mixture that is mainly composed of polysaccharides. To separate the active material from Antrodia camphorata mycelium, the present invention also provides a process, which includes using a unique liquid culture medium to grow Antrodia camphorata mycelium and separate the active material, and further gives a composition that contains the said active material.

In summary, the present invention provides a process to produce a kind of biologically active material from Antrodia camphorata mycelium and includes the procedures to grow Antrodia camphorata mycelium with a unique culture medium and to separate the active material for the said process.

The Antrodia camphorata mycelium used in the present invention is the CCRC 35398 and CCRC 35396 from Culture Collection and Research Center of Food Industry Research and Development Institute, Hsinchu, Taiwan, R.O.C.

The liquid culturing of Antrodia camphorata mycelium is described as follows. Place mycelium on a plate for culturing at a proper temperature, e.g. 15-35° C. and preferably at surrounding temperature of 25° C., for two weeks. Collect mycelium and place it into a beaker. Perform culturing with the listed culture medium in examples at about 30° C., pH 2-8, preferably pH 4-7, more preferably pH 4.5, and vibration speed 50-250 rpm until initial log period, i.e. 5-7 days. Finally, transfer the cultured species to fermentation tank containing culture medium (same as that in beaker). Perform culturing for 8-16 days at 15-30° C. (preferably at surrounding temperature 25° C.), a tank pressure of 0.1-1.5 kg/cm², pH below 4.5, with input of air or mixture of air, oxygen and carbon dioxide or nitrogen at 0.5-1.0 vvm, preferably air, under agitation at 50-300 rpm. The derived suspension culture solution for Antrodia camphorata mycelium contains mycelium and the supernatant.

Next, proceed with the procedures to separate the active material from the derived suspension culture solution.

The present invention includes two separation methods. One is to separate mycelium and supernatant from culture suspension of Antrodia camphorata, followed by separating active material respectively from the two parts. The other is to directly separate the active material from culture suspension of Antrodia camphorata, which includes mycelium and liquid culture medium.

The first separation method includes procedures to separate Antrodia camphorata mycelium and liquid and to extract the active material from mycelium and supernatant.

The separation can be performed by the traditional technology, such as centrifugation, grouptling and decantation etc. One of the preferred examples is to use centrifugation, such as European Centrifugation Dewatering Machine or Decater NX418 S from a Sweden company, ALFA LAVAL at 3200 rpm (4000×g) to separate out mycelium and supernatant.

The next is about procedures to separate the active material from mycelium and supernatant respectively. The ways to separate the active material from mycelium include solvent extraction and dissolution and re-separation of mycelium etc. Depending on the characteristics of the active material from Antrodia camphorata, convenience and industrial feasibility, the preferred way is solvent extraction. The preferred solvent is water, alkaline water or acidic water or mixture of the said solvents. In a preferred embodiment, water is used for extraction, which can be performed at temperature below 120° C. When water is used as the solvent for extraction, the temperature can be 30-120° C. and extraction solution is separated out after 30 minutes to 2 hours. Extraction can be repeated for several times and extraction solutions can be treated together.

The methods to separate the active material from extraction solution of mycelium and supernatant are identical, as described in the following. Concentrate the supernatant of culture solution by several times, such as 5-30 times, preferably 10 times, e.g. from 200 liters to 20 liters. Settle overnight with alcohol or alcohol/water, like 95% alcohol/water, at low temperature such as 0-30° C., preferably 4° C. Finally, separate the precipitates to obtain the desired active material.

For the other method in the present invention, it is to directly separate the active material from the culture suspension of Antrodia camphorata mycelium. It is to directly heat the culture solution including mycelium and culture medium to 30-121° C. for a certain period of time, like 30 minutes to 2 hours, followed by separating Antrodia camphorata mycelium. Then use above-mentioned procedures to separate the active material from supernatant.

In the second part of the present invention, the active material separated from culture suspension of Antrodia camphorata mycelium by the above-mentioned separation method is proposed. The biologically active material derived by the method in the present invention is mainly polysaccharide.

From previous study, it is known that the physiologically active material in mushroom is mainly the soluble polysaccharides. In the past, the source for mushroom polysaccharides is mainly from and limited to fruiting body extract. While liquid culturing of mycelium can produce polysaccharides exterior to cells in significant yield. Mycelium polysaccharides can inhibit the growth of cancer cells by improving host immunity. The study of the function for mushroom polysaccharides is as follows.

1. Structure Analysis

The raw polysaccharides extracted from mycelium and fruiting body by hot water is purified to dextran, hetero-polysaccharides and protein polysaccharides. After refinery by gel filtration several instrument analytical methods like chromatography, NMR spectra, IR spectra and GC-MS are used to analyze molecular weight, molecular bonding, branching degree and specific rotation for dextran and hetero-polysaccharides. The main structure like β-(1,3)-D-dextran, semi-lactose-β-dextran and α-mannose can be analyzed by x-ray diffraction and related to its medical function. For example, β-(1,3)-D-dextran appears in helical structure, which is possibly important to resist tumors. Because not all the mushroom polysaccharides have the activity to resist tumors, and their activity depends on water solubility, molecular weight, molecular conformation and branching degree. It is expected that chemical analysis can lead to finding the molecular mechanism to inhibit cancers.

It is known that the polysaccharide from baisdiomycete and with β-1, 3-dextran in backbone and β-1, 6-dextran in side chain shows significant difference in molecular weight distribution and in physiological activity. In general, it is divided according to molecular weight into (A) 3-5×10³ D, which can lower blood glucose such as ganoderan; (B) 10-1000×10³ D, which can provide anti-infection function; (C) above 30×10³ D, which can provide anti-tumor function, such as mushroom polysaccharides, Reishi mushroom polysaccharides and schizophyllum commune polysaccharides. Therefore, the molecular weight of the polysaccharides in the present invention is also determined to investigate the physiological activity.

It is known from literatures that mushroom polysaccharides have various kinds of biological activities, which include:

1. Anti-Tumor Activity:

In 1968, Japanese Ikegawa etc. proved by “Sarcoma 180/little white mouse belly medicine application or oral medicine application” that the extract from polyporaceae and fruiting body of edible mushroom by hot water could provide remarkable anti-tumor effect and complete tumor-reduction rate. Afterward, many researchers also proved that extract mainly containing polysaccharides could show satisfactory anti-tumor effect, complete tumor-reduction rate and low death rate.

Besides water-soluble β-1,3-dextran, mushroom contains salt-extracted or base-extracted β-polysaccharides of hetero-polysaccharides, like xylose, mannose, galactose and aldose etc., and protein complexes. Such hetero-polysaccharides show good anti-cancer effect by injection or oral medicine application.

2. Other Physiological Function Regulating Material:

The abilities to lower blood pressure, reduce cholesterol, immunity regulation, lower blood glucose activity and inhibit aggregation of blood platelets are all considered as important discoveries.

In the third part of the present invention, a composition is proposed to contain the active material from Antrodia camphorata in the present invention, proper diluent, excipients or support.

In the composition for the present invention, the suitable diluents are polar solvents, such as water, alcohol, ketones, esters and mixtures of the above solvents, preferably water, alcohol and water/alcohol mixture. For the preferable embodiment, the suitable solvents are water, normal saline, buffering aqueous solution and buffering saline etc. The excipients or supports, which may or may not exist in the composition for the present invention, can be in liquid or solid form, such as lactose, dextrin, and starch and sodium stearate. Liquid excipients include water, soybean oil, wine and juices etc.

The following examples serve to exemplify the present invention but do not intend to limit the scope of the present invention.

### EXAMPLE 1

### Tests with Antrodia camphorata Mycelium (CCCRC 35398) Culturing of Mycelium

Mycelium Fungus: CCRC35398 fungus preserved in Food Industry Research and Development Institute.

Plate Culture: Seed mycelium on plate and maintain at 30° C. for two weeks.

Beaker Culture: collect fungus grown on plate to put in beaker. Use the following culture medium at about 30° C. and pH 4.5 with vibrator operation at 50-250 rpm until initial log period, i.e. About 5-7 days.

Culture Medium Formula

| Components | Content (weight %) |
|---|---|
| Cereals (like flour) | 1 |
| Egg white | 0.1 |
| Magnesium Sulfate | 0.05 |
| Potassium hydrogen phosphate | 0.05 |
| Ferric Sulfate | 0.05 |
| Sucrose | 2 |
| Enzyme Extract, Powder, paste | 0.5 |
| Beans (like soy bean powder, green bean powder | 0.2 |
| etc.) | |

Fermentation Tank Culture:

The culture medium used is the same as above. The species grown in beaker is transferred to the fermentation tank, which is purged by air at 150 liter/min at 30° C., tank pressure 0.5-1.0 kg/cm² and pH below 4.5 with agitation of 200 rpm for about 10 days. The derived suspension of Antrodia camphorata culture includes the mycelium and the clear supernatant.

Result: 100 fermentation solution can be used to produce 2 kg mycelium (in dry state) and 90 liter supernatant.

### EXAMPLE 2

Separation of the Active Material from Antrodia camphorata

Separation of the Active Material respectively from Mycelium and Supernatant

Centrifugation is used to separate mycelium and supernatant. Traditional centrifugation machine of Decater NX418 S from Sweden ALFA LAVAL is operated at 3200 rpm (4000×g) to separate mycelium and supernatant.

Separation of the Active Material from Mycelium

Water at 80° C. is used for extraction for one hour. Then the extract is separated. Extraction can be repeated for several times. Extracted solutions are treated together.

Separation of the Active Material from Mycelium Extracted Solution and Cilture Supernatant

Concentrate the culture supernatant by ten times. Settle by 95% alcohol/water at 4° C. overnight to separate the active material from the precipitates.

Direct Separation of the Active Material from the Liquid Culture Suspension for Antrodia camphorata Mycelium

Direct heat the culture suspension containing mycelium and culture medium up to 100° C. for about one hour to separate Antrodia camphorata mycelium. Then separate the active material from supernatant by the above procedures.

Results:

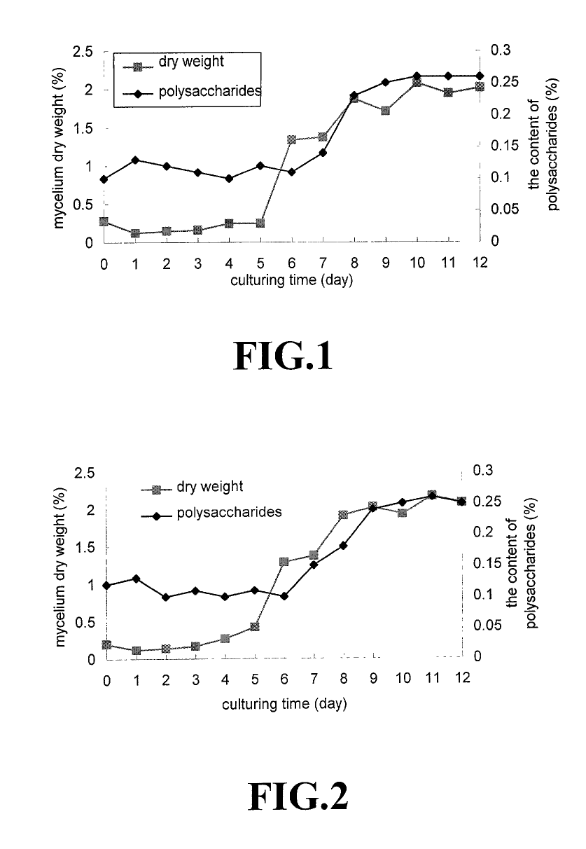

The yield for the active material is shown in FIG. 1. It is found that six days after culturing both dry weight and polysaccharide yield increase and reach to a stable state after ten days.

### EXAMPLE 3

### Tests with Antrodia camphorata Mycelium (CCCRC 35396)

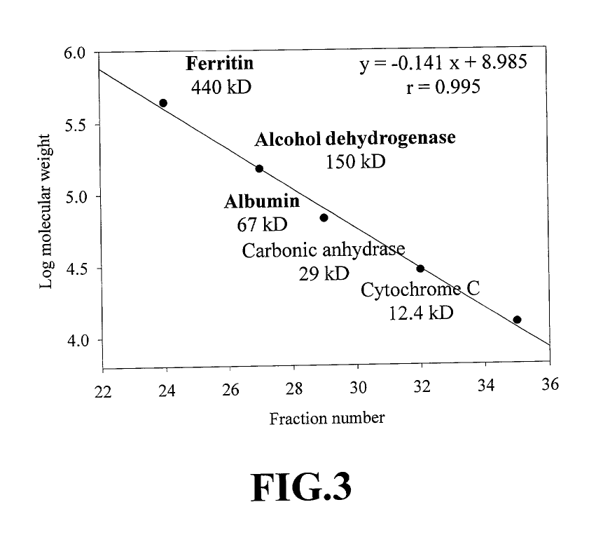

Perform tests by the same procedures as in Example 1 and Example 2 on another Antrodia camphorata mycelium (CCCRC 35396). For dry weight and polysaccharides, the results are similar to another culture (CCRC35398), as shown in FIG. 2. For dry weight, 100 liters of fermentation solution can produce 2±0.2 kg (dry weight) mycelium after fermentation and 90 liters of filtrate. For polysaccharides, as shown in FIG. 2, it is found that six days after culturing dry weight and polysaccharide yield apparently increase and reach to a stable state after ten days.

### EXAMPLE 4

### Active Material Analysis

I.Material and Process

1. Culture Preparation

Antrodia camphorata mycelium CCRC 35398 is purchased from Culture Preservation Center of Food Industry Research and Development Institute, Hsinchu, Taiwan, R.O.C. and cultivated by slope culture medium of potato dextrose agar (PDA) (purchased from Difco USA) and then stored.

2. Culture of Mycelium

Use in-depth culture process continuously for seven days at temperature 30° C. The culture quantity accounts for 1.0% of culture medium. Each liter of deionized water contains 20 g sucrose, 3 g (NHI4) 2SO4, 3 g MgSO₄, 3 g KH₂PO₄, 0.5 g citric acid, 5 g enzyme extract. The pH of culture solution is adjusted to 5.5.

3. Chemical Reagents

Alcohols, normal hexane and ethyl acetate (GR grade, from German Merck) and anhydrous sodium sulfate.

4. Extractions and Identification of Mycelium Composition

(1) Extraction

200 g of freeze dried Antrodia camphorata mycelium powder is heated, agitated, refluxed and extracted in 2 liters of methanol for five hours, followed by filtration. The residues are subject to the above-mentioned procedure repeatedly for two times. Combine the collected filtrate and concentrate it (40° C., 50 mTorr) under reduced pressure to obtain the concentrate (60.67 g).

(2) Identification

Place concentrate (60.0 g) and silica gel* (200 g) into vacuum evaporator for mixing. Take 20 g of mixture and load it to silica gel column (filled with 550 g of silica gel). Use the following solvents in 1000 ml for identification.

| Stripping | 1 | 2 | 3 | 4 | 5 | 6 | 7 | 8 |
|---|---|---|---|---|---|---|---|---|
| n-hexane (%) | 100 | 75 | 50 | 75 | 100 | 75 | 50 | 0 |
| Ethyl acetate | 0 | 25 | 50 | 75 | 100 | 75 | 50 | 0 |
| (%) | | | | | | | | |
| Methanol (%) | 0 | 0 | 0 | 0 | 0 | 25 | 50 | 100 |
| Total volume | 1000 | 1000 | 1000 | 1000 | 1000 | 1000 | 1000 | 1000 |
| (ml) | | | | | | | | |

*Silica gel (silica gel 60, 0.063˜0.200 mm, Merck)
Silica gel column (silica gel, 500 g, and column diameter: 80 cm × 4.5 cm)

II. Composition Analysis for Polysaccharides

Extraction Rate of Antrodia camphorata Polysaccharides

The highest extraction rate (14.33%) appears for the fermentation solution. The next highest extraction rate appears for the water extract of mycelium (2.98%). While the base extract of mycelium shows the lowest extraction rate (1.29%). The filtrate of mycelium polysaccharides apparently has higher extraction rate than water extract and base extract, which indicates mycelium polysaccharides are produced more outside cells than inside cells (Table 1).

For polysaccharides of Antrodia camphorata mycelium, since polysaccharides from the fermentation filtrate have 9.55% water content, while the polysaccharides for water extract and base extract have 10.75% and 4.35% respectively. Determined by phenol-sulfuric acid method, the filtrate has most polysaccharide content (87.15%), which is apparently higher than those of water extract (72.86%) and base extract (40.65%). This indicates a significant amount impurity exists in the base extract. Because some base-soluble inorganic salts and proteins are soluble in base extraction process, there are relatively high percentages of ash content (4.86%) and protein (14.18%).

TABLE 1
Polysaccharide Extraction Rate for Antrodia camphorata Mycelium

| Antrodia camphorata | % Extraction rate (w/w)¹ |
|---|---|
| Filtrate extract | 14.33 |
| Water extract | 2.98 |
| Noah extract | 1.29 |

¹average value (p < 0.05)

Analysis on Glucose Composition in Antrodia camphorata

The polysaccharide of Antrodia camphorata mycelium is under hydrolysis by 2M trifluoroacetate. Then use 1 N NaOH to neutralize it until pH is neutral. The decomposition of polysaccharides provides information on its composition (Table 2). The polysaccharide of fermentation filtrate is mainly composed of mannose (188.54 mg/g), glucose (150.11 mg/g) and xylose (112.75 mg/g). While water extract is mainly composed of glucose (355.77 mg/g), xylose (205.30 g/mg) and galactose (121.39 mg/g). Base extract is composed of glucose (177.11 mg/g) and xylose (147.23 mg/g), which still has a little glucose and aldose acid. The water extract has most aldose acid (102.40 mg/g). The next is base extract (68.56 mg/g) and fermentation filtrate (54.72 mg/g).

TABLE 2
Glucose Compositions for Polysaccharide Extract from Antrodia
camphorata Mycelium Fermentation Solution

| | Content¹ (mg/g dried sample) | | |
|---|---|---|---|
| Glycogen | Filtrate extract | Water extract | NaOH extract |
| Ribose | N.D.² | N.D. | 13.41 |
| Xylose | 112.75 | 205.30 | 147.23 |
| Mannose | 188.5 | N.D. | N.D. |
| Glucose | 150.11 | 355.77 | 177.11 |
| Galactose | 88.44 | 121.39 | 52.00 |
| Aldose acid | 54.72 | 102.40 | 68.56 |

¹Same values in column with different alphabet have different statistical meanings (p < 0.05).
²N.D.: not determined.

Multiple Molecule Inspection of Glycogen

Molecular Weight Determination for Polysaccharides

Preparation for Protein Standard Curve of Gel Filtration Chromatography

Column: Spectra/chrom LC column (1.6×70 cm)

Gel: Sepharose®6B

Mobile Phase: 0.15M NaCl

Flow Rate: 0.5 ml/minute, 3.0 ml/column

Polysaccharide: phenol-H₂SO₄ method, UV 480 nm

Protein: measured at 254 nm

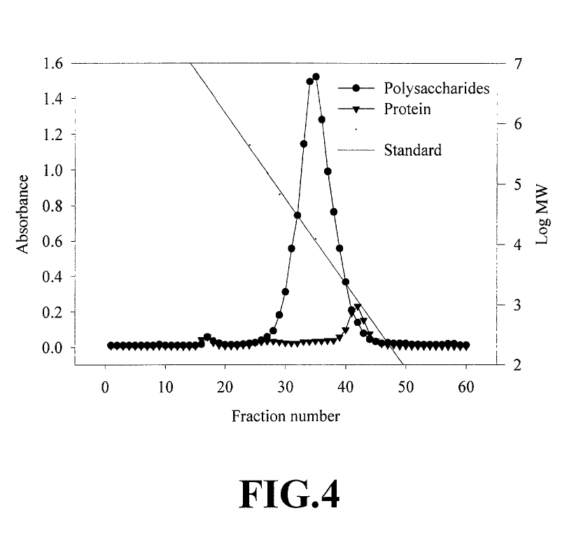

Sepharose®6B is a commercial product in the form of gel of 6% Agarose and suitable for molecular weight determination for 10⁴˜10⁶ polysaccharide molecule and 10⁴˜4×10⁶ protein molecule. Its column volume is determined by Blue dextran as 45 ml. Protein standards of different molecular weights include ferritin (MW 4.4×10⁵ Da), de-alcohol hydrogen (MW 1.5×10⁵ Da), egg white (MW 4.7×10⁴ Da), carbonic anhydrase (MW 2.9×10⁴ Da) and cell colorant C (MW 1.24×10⁴ Da). After the standards pass through column Sepharose®6B, the log values of standard molecular weights are plotted against tube numbers. An initial regression line is also derived. FIG. 3 shows the protein standard curve for gel filtration chromatography.

Molecular Weight Determination of Polysaccharides

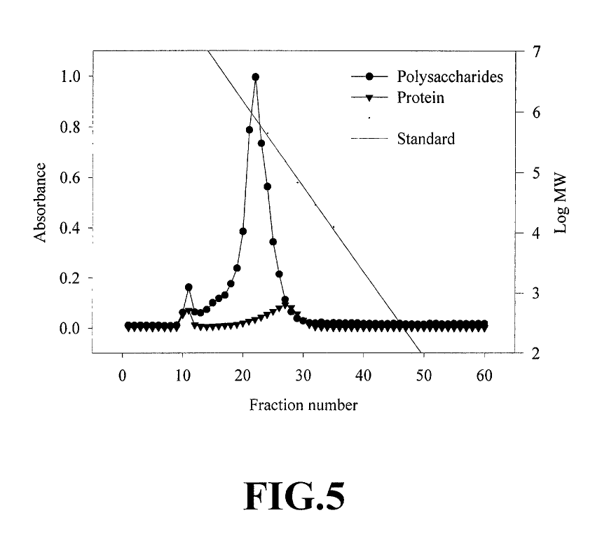

Under identical conditions, samples undergo gel filtration chromatography. The maximum absorbance for proteins at wavelength of 254 nm is used to determine the tube numbers. Phenol-sulfuric acid is used to display color. The tube numbers for the color-displaying samples are used with the regression line to determine the molecular weight of polysaccharide, as shown in FIG. 4.

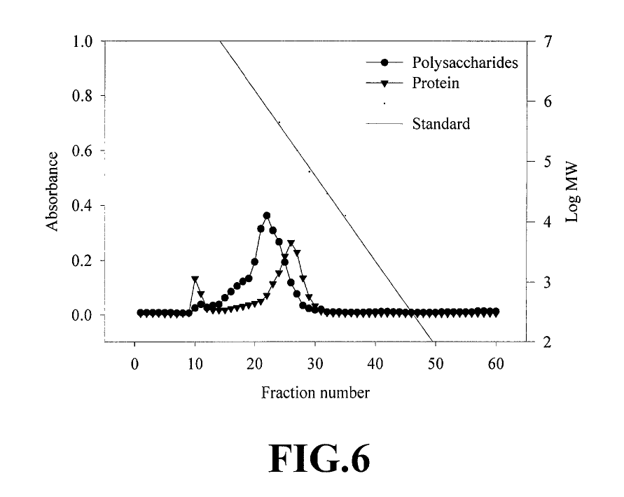

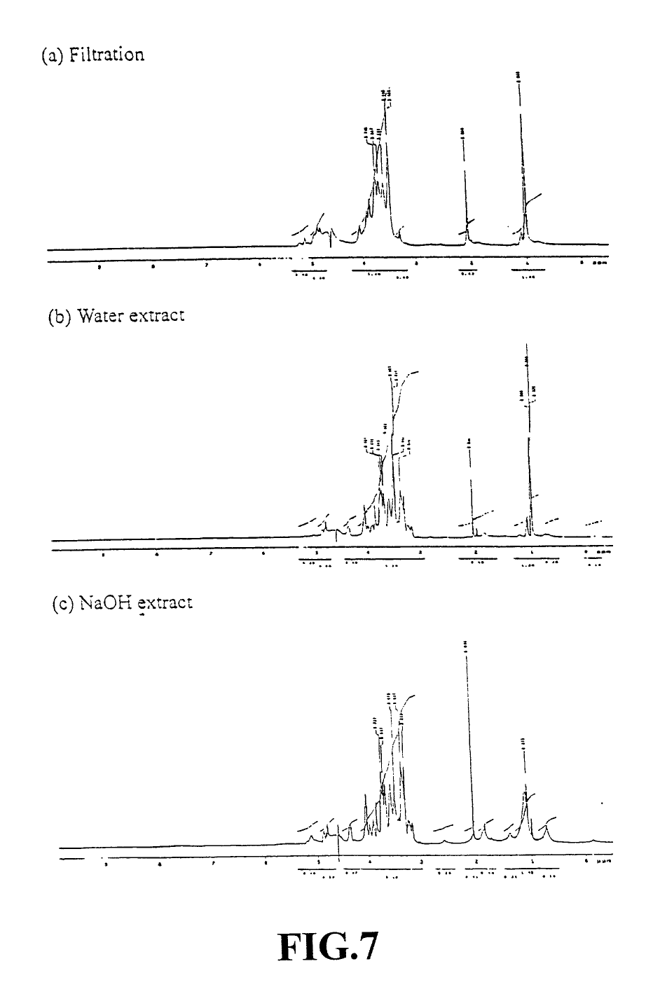

After separation and color displaying by sulfuric acid method, it is known that absorbance peaks appear at tube number 17 and tube number 35 for polysaccharide fermentation filtrate (FIG. 4). After comparison to standards (FIG. 3), it is found that the molecular weights of polysaccharides are above 10⁶ Da and 1.1×10⁴ Da. Both water extract and base extract show absorbance peaks at tube number 11 and 22 (FIG. 5 and FIG. 6). After comparison to standards, they have polysaccharide molecules of more than 10⁶ Da and 7.6×10⁵ Da, which indicates it may contain β-1, 3-D-furan dextran of molecular weight 50˜200×10⁴ with β-1, 6-glucose side chain.

Structure Analysis for Polysaccharides in Antrodia camphorata

In nature, polysaccharides are polymers of aldose or ketose with glycosidic linkage, a necessary part for living organism, showing anti-tumor characteristic in fungi. Usually polysaccharides link with proteins to form glycoprotein, which attracts attention for its anti-tumor activity. Some researchers have separate complex compound of β-1, 6-dextran and protein from Agarics (polysaccharide: protein=50:40). Besides proflamin, active glycoprotein from golden mushroom, is composed of 10% glucose and 90% protein with molecular weight 13000±4000 Da. It shows clear inhibition effect to tumor B-6 or cancer 755. The anti-tumor active β-polysaccharide EA6 (glucose: protein 70:30) extracted from fruiting body has been proved to have the antibody activity related to host media anti-cancer characteristic. Hence the ratio for polysaccharide to protein needs to be investigated for anti-tumor activity and structure analysis.

1. NMR Analysis

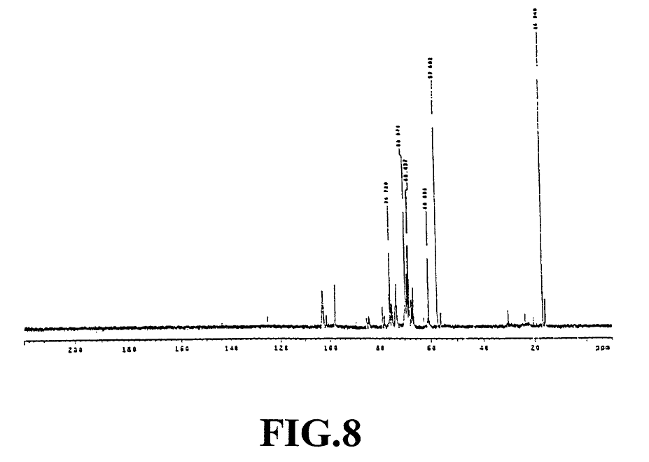

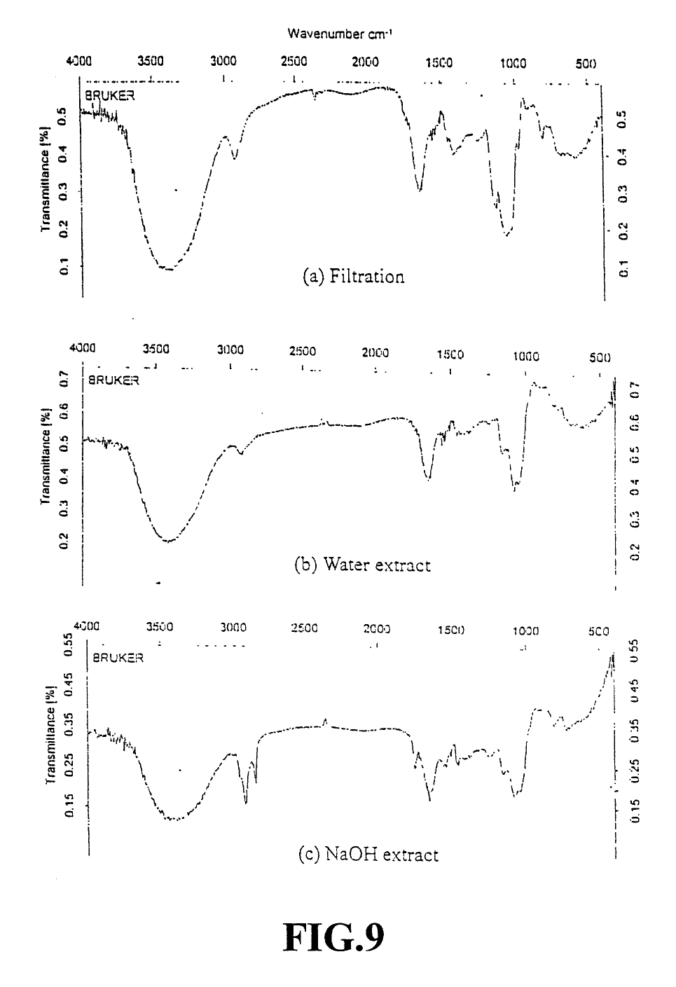

¹H-NMR chemical shift for β-D-dextran of Antrodia camphorata mycelium at 3˜4 ppm is the hydrogen on carbon bonding. Chemical shifts for fermentation filtrate are 4.570 (H1), 4.063 (H-6a), 3.866 (H-6b), 3.687 (H-5), 3.496 (H-4), 3.486 (H-3) and 3.303 (H-2) (FIG. 7). The hydrogen NMR spectra results for water extract and base extract are similar with chemical shifts 4.570, 4.598 (H-1), 4.034, 4.036 (H-6a), 3.837 (H-6b), 3.662, 3.660 (H-5), 3.454, 3.473 (H-3,4) and 3.336, 3.337 (H-2), which corresponding C13 spectra chemical shifts are 103.087 (C-1), 78.775 (C-3), 77.978 (C-5), 76.092 (C-2), 73.224 (C-4) and 75.505 (C-6) (FIG. 8). The results are similar to those from Mizuno etc. on one-dimensional hydrogen spectra chemical shift for water-soluble polysaccharides in mushroom fruiting body.

2. IR Analysis

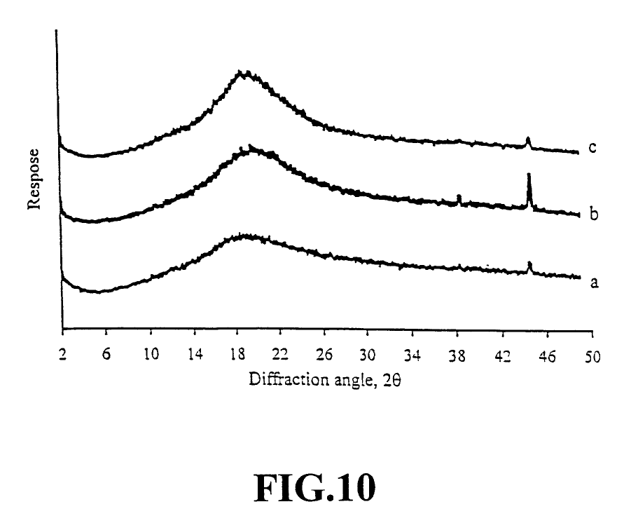

Powder of Antrodia camphorata mycelium is subject to IR analysis. Fermentation filtrate indicates OH group at 3375 cm⁻¹, W shape peaks at 1557 cm⁻¹, which means C—C—C bonding exists. C—H group is found at 2938 cm⁻¹, and —CH—O—CH— is found at 1063 cm⁻¹ (FIG. 9). Water extract and base extract polysaccharides indicate W shape peaks at 3419, 3390 cm⁻¹ (OH group), 1557, 1539 cm⁻¹ (C—C—C) and absorbance bands at 2922, 2919 cm¹ (C—H) and 1080, 1069 cm⁻¹ (—CH—O—CH—), which indicates mycelium polysaccharides have characteristics of polysaccharide groups.

3. X-Ray Diffraction Analysis

X-ray diffraction pattern for the extract of Antrodia camphorata mycelium shows 2θ angle at 19.43° for fermentation filtrate and 19.48°, 19.37° for water extract and base extract respectively (FIG. 10). From this figure, it is shown that better degree of crystallization exits in base extract than in water extract or filtrate extract.

### EXAMPLE 5

### Activity Analysis

Improve Immunity

A. Activation Test on Macrophage

Test Culture: Antrodia camphorata CCRC 35398 and CCRC 35396

Test Method:

Sample Preparation

Follow the above-mentioned procedures for fermentation. Then use centrifugation to obtain mycelium and fermentation solution. Use hot water (above 100° C.) and alkaline solution (NaOH) to extract on mycelium. The three obtained extracts (mycelium water extract, mycelium base extract and fermentation solution) are extracted for polysaccharides by alcohol. Finally, freeze-dry the extracted polysaccharides. Three freeze-dried products are dissolved by double distilled sterile water to concentration of 10 mg/ml to form extract solution of Antrodia camphorata polysaccharide.

Activation Test:

Add J774A.1 macrophage (CCRC60140) in 1×10⁵ cells/pore into the three prepared extract solutions of Antrodia camphorata polysaccharide for activation test. The final concentration is 100 μg/ml. Each sample repeats the test for three times. Take out cell culture solution the next day. Use ELISA method to analyze the TNF-a concentration from macrophage.

Group:

(a) Negative Reference—add 2 μl of phosphate buffering solution to macrophage for activation.

(b) Positive Reference—add 2 μl of lipopolysaccharide (LPS, final concentration 10 μg/ml) to macrophage for activation.

(c) Experiment—add 2 μl of different Antrodia camphorata extracts to macrophage for activation until final concentration of 100 μg/ml.

Result:

Tumor necrotic factor (TNF-α) has the functions to destroy tumor cells and activate immune cells. So it plays an important factor in immune system. The result is shown in FIG. 3. Three experiment groups have apparently higher TNF-α concentration than negative reference group. Wherein, base extract of Antrodia camphorata mycelium has the highest, but still lower than that of positive reference group. Hence, test result shows all extracts from Antrodia camphorata can stimulate and activate macrophage. Base extract is the most effective one.

B. Analysis and Evaluation on Immune Function of Antrodia camphorata Active Material under Live Animal Test

The experiment uses BALB/cByJ little mice as the experimental animals. Oral administration is used for five weeks. Various immune functions of spleen cells are analyzed to evaluate the effect of Antrodia camphorata mycelium on immune response regulation.

Five weeks after feeding, it has no effect on little mice. Use MTT to perform analysis on lymphocytic cell increase. It is found that under ConA and PHA treatment it promotes lymphocytic cell increase. Under ConA stimulation spleen is stimulated to produce Th1 cytokine IL-2, but inhibited to form Th2 cytokine IL-4.

Material and Process

1. Experimental Animal

Six weeks old, female BALB/cByJ little mice, SPF grade, purchased from National Laboratory Animal Breeding and Research Center.

After purchase, animals are monitored for one week to evaluate their health and growth. If any abnormal situations happen (fear of light, dehydration), abandon the mouse.

Weigh the mouse before experiment and abandon those of weight excluded to the range (average weight±standard deviation). The qualified mice are made into three groups. Each group has the same gender and 12 mice. Ear tag is used for identification. Weigh the mice once every week to investigate their growth.

2. Feeding and Caring

Follow conventional feeding and caring methods for experimental animals. Animal incubation room is set at 23±2° C., 50±10% relative humidity with 12 hours of light exposure/dark schedule and no limit on feeding water.

3. Experiment Sample

Antrodia camphorata mycelium (CCRC 35396) undergoes fermentation as in Example 1, followed by processing and drying to form samples (lot number: 20020315A9B).

4. Dosage Design

The experiment proceeds with a reference group and two test groups. The dosage for test groups is calculated according to human daily dosage for little mice. Enlarge the dosage by ten times as the high dosage group.

I. Reference—equal volume of second distilled water

II. Low Dosage—daily suggested quantity

III. High Dosage—ten times of daily suggested quantity

Dosage calculation is as follows:

Suggested for Normal Person: 420 mg/tablet×2 tablets/time×3 times/day=2520 mg/day

Hence, conversion to dosage of little mice is 2520 mg/day×0.0026=6.552 mg/day for low dosage group. While high dosage group is 65.52 mg/day (6.552 mg/day×10).

5. Animal Feeding Method and Days

Use stomach tube and oral administration for feeding. Once daily. Six days per week for continuous five weeks.

6. Experiment Procedures

6.1 Animal Blood Sampling and Sacrifice After experimental animals are subject to Euthansia by CO₂ and died, their bodies are sprayed with alcohol for disinfecting, followed by sterile operation in Laminar flow and spleen removal.

6.2 Preparation of Spleen Cell Suspension

Under sterile condition, take spleens from mouse bodies. Place them in petri dishes in 30 ml containing 5-ml culture medium. Use the flat end of needle syringe to hold spleen and rub until whole spleens turn into white and make cells among connective tissue releasing out as much as possible.

Use sterile pipette to draw culture medium containing cells into 15-ml centrifugation tube. Rest for 5˜10 minutes. Draw cell suspension to another centrifugation tubes and start centrifugation under 600×g for five minutes. Discard the supernatant. Gently flap the tube wall to evenly disperse the cells. Add 5-ml icy ACK RBC lysis buffer to mix with cells for one minute. Immediately add 5 ml warmed culture medium. Perform centrifugation for five minutes. Discard the supernatant. Gently flap the tube wall to evenly disperse cells. Rinse with 10 ml HBSS buffer twice. Place cell suspension in 10-ml culture medium and dilute with Trypan Blue (about ten times). Calculate the total number of cells. Adjust concentration of cells by culture medium to 1×10⁷ cells/ml.

6.3 Lymphocytic Cell Increase (MTT Method)

Add 100 μl/pore culture medium or culture medium containing mitogen (10 μg/ml ConA, 20 μg/ml PHA and 50 μg/ml LPS) to the 96-pore culture dish. Then add 100 μl/pore with 4×10⁶ cells/ml spleen cell suspension in 37° C., 5% CO₂ culture box for 72 hours.

After culturing, add 20 μl/pore MTT (5 μg/ml) for another four hours. Perform centrifugation at 250×g for ten minutes. Discard the supernatant in 2001/pore. Add 200 μl/pore DMSO for vibration for five minutes. Use ELISA reader to test A₅₇₀ nm.

6.4 Cytokine Test

Label “cell only” and “treated by ConA” on 24-pore culture dish. Add 0.6 ml culture medium to “cell only” pore and 0.5 ml ConA (10 Lig/ml) and 0.1 ml culture medium to ConA treated pore. Add 0.4 ml of mice spleen cells with 10⁷ cells/ml to each pore. After 24 hours, collect the supernatant and place it in 20° C. refrigerator. Use sandwich-ELISA (enzyme-linked Immunosorbent assay) to determine IL-2 and IL-4 content in cell culture supernatant.

7. Data Processing and Evaluation on Results

The experimental result is expressed by Mean±SD. All data are analyzed statistically by one-way ANOVA. Compare among each group by Duncan's multiple range tests. Use Dunnett's t-test to compare experiment groups against reference group.

Result

After feeding Antrodia camphorata mycelium for five weeks, no apparent difference in growth exists among reference group, low dosage group, and high dosage group by comparing the mouse weight (Table 3). This indicates Antrodia camphorata mycelium has no adverse effect on mouse growth.

Spleen cells are treated by ConA, PHA and LPS mitogen under 5% CO₂ at 37° C. for three days. Use MTT to analyze lymphocytic cell increase. It is found that Antrodia camphorata mycelium can significantly stimulate lymphocytic cell increase (P<0.05 and <0.1) (Table 4) under the stimulation by ConA and PHA.

Under self-induction situation (i.e. cell only) and the stimulation by ConA mitogen, spleen is treated under 5% CO₂ at 37° C. for 24 hours. Collect the supernatant. Analyze respectively the quantity of grown IL-2 and IL-4 to understand the effect of Antrodia camphorata mycelium on cytokine. The result shows that Antrodia camphorata mycelium can stimulate IL-2 Cytokine of Th1-type (ConA-Stimulated), while inhibit IL-4 Cytokine of Th2-type (ConA-Stimulated) (Table 5).

Conclusion

After five weeks of feeding Antrodia camphorata mycelium, no apparent difference exists among low dosage, high dosage and reference. Under the stimulation of ConA and PHA, Antrodia camphorata mycelium can increase lymphocytic cells and promotes the increase of IL-2 Cytokine of Th1-type by Spleen cells and inhibits the increase of IL-4 Cytokine of Th2-type.

TABLE 3
Average Weight of Little Mice during Experiment Period

| | Reference | Low dosage | High dosage |
|---|---|---|---|
| Week | 12 mice/group | 12 mice/group | 12 mice/group |
| 1 | 20.34 ± 1.86 | 20.12 ± 1.52 | 20.81 ± 1.37 |
| 2 | 22.47 ± 1.68 | 22.99 ± 1.24 | 22.57 ± 1.88 |
| 3 | 24.34 ± 1.81 | 24.38 ± 1.22 | 24.75 ± 1.85 |
| 4 | 26.08 ± 1.55 | 25.81 ± 1.39 | 25.78 ± 1.84 |
| 5 | 27.08 ± 1.96 | 26.64 ± 1.35 | 26.59 ± 1.68 |

1. The Antrodia camphorata mycelium for five weeks of feeding is derived from Antrodia camphorata (CCRC35396) that has undergone fermentation, treatment and drying.
2. The result is expressed in Mean ± SD with weight unit in gm.

TABLE 4
Effect of Antrodia camphorata Mycelium
on Increase of Lymphocytic Cells

| | | Reference | Low dosage | High dosage |
|---|---|---|---|---|
| Stimulation | ConA | 4.40 ± 1.74 | 9.81 ± 2.44 | 4.69 ± 1.94 |
| index | PHA | 3.71 ± 0.70 | 4.53 ± 1.11 | 3.94 ± 1.34 |
| | LPS | 5.82 ± 2.92 | 5.77 ± 1.71 | 4.98 ± 1.66 |

1. The Antrodia camphorata mycelium for five weeks of feeding is derived from Antrodia camphorata (CCRC35396) that has undergone fermentation, treatment and drying.
2. The result is expressed in Mean ± SD with weight unit in gm.
3. *P < 0.05, **P < 0.01

TABLE 5
Effect of “Antrodia camphorata King” on Spleen Cell Cytokine

| Cytokine | Group treatment | Reference | Low Dosage | High Dosage |
|---|---|---|---|---|
| IL-2 | No treatment (Cell only) | 5.53 ± 2.19 | 4.80 ± 2.62 | 6.89 ± 1.64 |
| (μg/mg) | ConA stimulation | 3233.5 ± 548.1 | 4400.8 ± 1782.3 | 5893.9 ± 1577.3* |
| IL-4 | No treatment (Cell only) | 3.35 ± 1.75 | 3.81 ± 2.23 | 4.66 ± 2.83 |
| (μg/mg) | ConA stimulation | 1142.7 ± 364.3 | 826.4 ± 220.2** | 1095.1 ± 499.7 |

1. The Antrodia camphorata mycelium for five weeks of feeding is derived from Antrodia camphorata (CCRC35396) that has undergone fermentation, treatment and drying.
2. The result is expressed in Mean ± SD with weight unit in gm.
3. *P < 0.05, **P < 0.01

### EXAMPLE 6

### Activity Analysis

Enhance Immunity

The active material of Antrodia camphorata can stimulate lymphocytic cells in normal human blood to produce Cytokine, which can kill U-937 human lymphocytic cancer cells (Table 6) and also increase phagocytosis ability (Table 7) of macrophage (J744A. 1).

TABLE 6
Inhibitions to Human Lymphocytic Cancer Cells by Hot Water-Soluble
Polysaccharides from Fruiting Body and Mycelium of
Antrodia camphorata

| Sample | Dosage (μg/ml) | Inhibition rate (%) |
|---|---|---|
| Fruiting Body of | 0 | 14.461 |
| Antrodia camphorata | 2 | 26.23 |
| | 20 | 43.87 |
| Mycelium of Antrodia camphorata | 0 | 15.196 |
| | 2 | 25.49 |
| | 20 | 23.53 |

TABLE 7
Effect of Antrodia camphorata Mycelium on Phagocytosis Ability of
Human Macrophage

| Sample | Dosage (μg/ml) | Phagocytosis Ability |
|---|---|---|
| Polysaccharide for Antrodia | 3.9 | 147 |
| camphorata Mycelium | 15.6 | 159 |
| Polysaccharide for Antrodia | 3.6 | 152 |
| camphorata Culture (I) | 15.6 | 203 |
| Polysaccharide for Antrodia | 3.9 | 242 |
| camphorata Culture (II) | | |
| Polysaccharide for Fermentation | 3.2 | 144 |
| Filtrate of Reishi Mushroom | 23.5 | 233 |
| Reference | — | 100 |

Macrophage Culture (J774A.1)

The present experiment shows that cytokine performance and activity are stimulated and enhanced after feeding of different dosages for different numbers of weeks. Live animal experiment further proves that immune activity of cytokine from the stimulation by Antrodia camphorata can offer medical effect in living species.

Please refer to FIG. 2 for the results that show the immune response (cytokine IL-2, TNF-α, INF-γ) of little mice of C57BL/6 and BALB/c that have been fed with different dosages for different weeks. In live animal evaluation model, we use two mice (C57/BL6 and BALB/c) for experiment. Mice of C57/BL6 and BALB/c of 8 weeks old are divided into several groups. Each group has ten little mice, each of which is fed with Antrodia camphorata for one, two and four weeks. Each group has oral administration dosage for 1.0 mg, 2.5 mg or 5.0 mg. 24 hours after each mouse has taken dosage, and about 150±10 infant Schitosoma Mansonis has spontaneously infected mice at tails. Little mice without taking dosage are simultaneously infected by the same number of infant Schitosoma Mansonis, which is used as reference group. After six to eight weeks, portal perfusion method is used to sacrifice the animals by purging out the grown Schitosoma Mansonis in anal veins and mesenteric veins. The experiment result shows two mice after taking 2.5 mg or 5.0 mg of Antrodia camphorata produce the grown Schitosoma Mansonis in a quantity that is not very different from that of the reference group. Two weeks after oral administration of 1.0 mg Antrodia camphorata, the obtained result is similar to the previous result. When the two mice have taken 2.5 mg for two weeks, the number of adults grown from infants in body's shows clear decrease compared to the reference group, which worm reduction rate is between 20% and 45%. For oral administration of 5.0 mg, the effect is more prominent for BALB/c little mice after two weeks than after one week (worm reduction rate 40% vs. 26%). But for C57BL/6 little mice, the effect for two weeks are similar to that for one week. But when two mice have orally taken 1.0 mg or 2.5 mg of Antrodia camphorata active material for four weeks, the number of grown worms are significantly decreased, compared to reference group. Especially for dosage of 2.5 mg, the worm reduction rate reaches to 60% and 49%. The research result shows that when 2.5 mg of Antrodia camphorata has been taken for four week, the enhanced immunity provides significant effect in body (indicating a decrease of half infection rate) (as shown in Table 8).

TABLE 8
Worm Reduction Rate for C57BL/6 and BALB/c Mice that Have Taken
Different Dosages of Antrodia camphorata for One, Two and Four Weeks

| Weeks | 1 | | | 2 | | | 3 | | |
|---|---|---|---|---|---|---|---|---|---|
| Dosage | 1.0 | 2.5 | 5.0 | 1.0 | 2.5 | 5.0 | 1.0 | 2.5 | 5.0 |
| mg Mice | | | | | | | | | |
| Worm | | | | | | | | | |
| reduction | | | | | | | | | |
| rate | | | | | | | | | |
| C57BL/6 | —* | 5% | 10% | 25% | 20% | 14% | 33% | 60% | — |
| BALB/c | — | 11% | 26% | 29% | 27% | 40% | 34% | 48% | — |

*Indicating no test is performed.

1. Process to prepare the active material from Antrodia camphorata mycelium, which includes the following procedures:
(1) Plate Culture: Seed mycelium on plate and maintain at 30° C. for two weeks.
(2) Beaker Culture: collect fungus grown on plate to put in beaker. Use the following culture medium at about 30° C. and pH 4.5 with vibrator operation at 50-250 rpm until initial log period, i.e. About 5-7 days;
Culture Medium Formula

| Components | Content (weight %) |
|---|---|
| Cereals (like flour) | 1 |
| Egg white | 0.1 |
| Magnesium Sulfate | 0.05 |
| Potassium hydrogenphosphate | 0.05 |
| Ferric Sulfate | 0.05 |
| Sucrose | 2 |
| Enzyme Extract, Powder, paste | 0.5 |
| Beans (like soy bean powder, green bean powder | 0.2 |
| etc.) | |

(3) Fermentation Tank Culture: Transfer the cultured species to fermentation tank containing culture medium (same as that in beaker). Perform culturing for about 10 days at 30° C., a tank pressure of 0.5-1.0 kg/cm², pH below 4.5, with input of air at 150 liter/minute, under agitation at 200 rpm to obtain suspension culture solution for Antrodia camphorata mycelium containing mycelium and supernatant;
(4) Centrifuge the solution from step (3) to separate out mycelium and supernatant;
(5) Use solvents to extract the biologically active material.

2. As described in claim 1 for a process to produce active material from Antrodia camphorata mycelium, wherein the mycelium is the Antrodia camphorata mycelium registered as CCRC 35398 and stored in Culture Collection and Research Center of Food Industry Research and Development Institute, Hsinchu, Taiwan, R.O.C.

3. As described in claim 1 for a process to produce active material from Antrodia camphorata mycelium, wherein the mycelium is the Antrodia camphorata mycelium registered as CCRC 35396 and stored in Culture Collection and Research Center of Food Industry Research and Development Institute, Hsinchu, Taiwan, R.O.C.

4. As described in claim 1 for a process to produce active material from Antrodia camphorata mycelium, wherein the separation procedures include separation of the culture suspension into solid mycelium and culture supernatant, followed by solvent extraction for the said mycelium, followed by the combination of the said extract and the supernatant, followed by the precipitation of the active material.

5. As described in claim 1 for a process to produce active material from Antrodia camphorata mycelium, wherein the extraction solvent is water and extraction temperature is 30° C. to 121° C.

6. As described in claim 1 for a process to produce active material from Antrodia camphorata mycelium, wherein the separation procedures include direct heating of culture suspension at 30° C. to 121° C., followed by precipitation and separation of the active material.

7. As described in claim 1 for a process to produce active material from Antrodia camphorata mycelium, wherein the biologically active material is derived from the entire culture suspension for Antrodia camphorata mycelium.

8. As described in claim 1 for a process to produce active material from Antrodia camphorata mycelium, wherein the biologically active material is derived from the entire culture suspension for supernatant.

9. As described in claim 1 for a process to produce active material from Antrodia camphorata mycelium, wherein the biologically active material is derived from the entire culture suspension for mycelium extract.

10. A kind of biologically active material from Antrodia camphorata mycelium, which is derived after culturing of Antrodia camphorata mycelium with the characteristic of significant amount of polysaccharides inside.

11. A composition that contains the biologically active material from Antrodia camphorata mycelium as in claim 10.

12. As described in claim 11 for the biologically active material from Antrodia camphorata mycelium, which can stimulate the increase of lymphocytic increase, promote the formation of IL-2 of Th1-type cytokine and provide inhibition to the formation of IL-4 of Th2-type cytokine, and stimulate and activate macrophage as well.
